(19) United States
(12) Patent Application Publication
(10) Pub. No.: US 2022/0313254 A1
(43) Pub. Date: Oct. 6, 2022

(54) A SURGICAL STAPLER AND COMPONENTS AND METHODS THEREFOR

(71) Applicant: BOLDER SURGICAL, LLC, Marlborough, MA (US)

(72) Inventors: Richard Granger, Marlborough, MA (US); Allison Lyle, Marlborough, MA (US); Nathan Steketee, Marlborough, MA (US)

(73) Assignee: BOLDER SURGICAL, LLC, Marlborough, MA (US)

(21) Appl. No.: 17/847,059

(22) Filed: Jun. 22, 2022

Related U.S. Application Data
(63) Continuation of application No. PCT/US2021/012253, filed on Jan. 6, 2021.
(60) Provisional application No. 62/958,784, filed on Jan. 9, 2020.

(51) Int. Cl. A61B 17/072; A61B 17/064

(52) U.S. Cl. CPC A61B 17/072; A61B 17/064; A61B 2017/07271; A61B 2017/07264

(57) ABSTRACT

A surgical stapler and related components and methods are disclosed. The stapler has an anvil having a staple pocket, and a cartridge. The cartridge supports a staple pusher and a B-form staple, the B-form staple having a backspan, a proximal leg, and a distal leg, the backspan having a proximal portion coupled to the proximal leg, a distal portion coupled to the distal leg, and a central portion therebetween. The surgical stapler is configured to cold work at least a portion of the backspan during a deployment of the surgical staple.

### RELATED APPLICATION DATA

This application is a continuation of International Patent Application No. PCT/US2021/012253 filed on Jan. 6, 2021, which claims priority to U.S. Provisional Application No. 62/958,784, filed on Jan. 9, 2020 and entitled “A Surgical Stapler and Components and Methods Therefor”. The entire disclosures of the above applications are expressly incorporated by reference herein.

### FIELD

This invention is related to surgical tools. Specifically, but not intended to limit the invention, embodiments of the invention are related to surgical staplers.

### BACKGROUND

A number of surgical staplers are known in the industry and used in laparoscopic procedures. Typically, the staplers are supplied with what is known as B-form staples, because the staples generally form a B-shape when deployed.

It is desirable to provide a surgical stapler and components that improve patient healing and reduce patient trauma during surgical procedures, along with other new and useful improvements.

### SUMMARY

An exemplary surgical stapler configured to apply a surgical staple to tissue has an anvil having a staple pocket and a cartridge. The cartridge supports a staple pusher and a B-form staple, the B-form staple having a backspan, a proximal leg, and a distal leg, the backspan having a proximal portion coupled to the proximal leg, a distal portion coupled to the distal leg, and a central portion therebetween. The surgical stapler is configured to cold work at least a portion of the backspan during a deployment of the surgical staple.

An exemplary method includes (a) providing a surgical stapler having an anvil and a cartridge, the cartridge supporting a staple pusher and a B-form staple, the B-form staple having a backspan and a pair of legs extending therefrom; (b) causing the staple pusher to approximate the staple toward the anvil: and (c) during the approximating, coldworking the backspan of the staple.

### BRIEF DESCRIPTION ON THE DRAWINGS

FIG. 1 is a partial side view of exemplary components of a stapler described herein;

FIG. 2 is a partial bottom view of an exemplary anvil;

FIG. 3 is a partial side view of exemplary components of a stapler described herein;

FIG. 3a is a detail view of the components illustrated in FIG. 3;

FIG. 4 is a side view photograph of staples formed using the exemplary staple pusher and anvil pocket illustrated in FIG. 3;

FIG. 5 is a side view photograph of staples formed using the exemplary staple pusher illustrated in FIG. 3 and a curved anvil pocket;

FIG. 6 is an illustration of prior art;

FIG. 7 is a side view photograph of notched staples formed using components described herein;

FIG. 8 is a flowchart of an exemplary method;

FIG. 9 is a flowchart of an exemplary method; and

FIG. 10 is a table entitled Vessel Leak Test Summary.

### DETAILED DESCRIPTION

By way of introducing the reader to the details of embodiments herein, those skilled in the art of medical devices and, more specifically, surgical staplers, are familiar with traditional configurations of surgical staplers. Typically, the surgical stapler includes an anvil and a cartridge which move toward one another to compress tissue therebetween. Thereafter, a staple pusher and staple housed in the cartridge are moved toward the anvil. The anvil typically has two staple pockets that deform the two respective legs of a B-form staple to form a B closure about the tissue.

Before turning to details of the device and methods disclosed herein, it is noted that, for the purpose of this document, absolute terms like “flat” shall be understood to mean within a reasonable manufacturing tolerance at the time of the writing of this document or at the time of manufacture. Additionally, the term “central” shall be understood not to mean a perfect center point of a feature, but rather between other points of the feature.

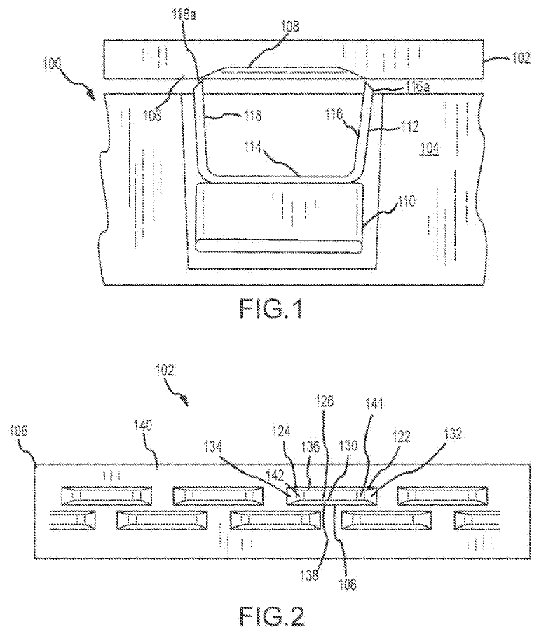

With reference to FIGS. 1-2, some embodiments described herein include a medical device 100 such as a surgical stapler having an anvil 102 and a cartridge 104 configured to cooperate to staple tissue positioned therebetween. In some embodiments, the device 100 may function substantially as described in commonly-assigned U.S. Pat. No. 10,143,474 entitled “Surgical Stapler,” and issued on Dec. 4, 2018, the entire disclosure of which is incorporated for all proper purposes. In some embodiments, the stapler described herein may include an end effector configured for engagement with a surgical stapler actuator, like that described in U.S. Pat. No. 10,143,474. In some embodiments, the end effector may comprise an anvil having a staple pocket, and a cartridge, wherein the cartridge is configured to support a staple pusher and to house a staple.

The anvil 102 may have a tissue interacting face 106 and at least one staple pocket 108. The anvil 102 may have a row of staple pockets 108. The anvil 102 may have a plurality of rows of staple pockets 108, as seen in FIG. 2.

Returning to FIG. 1, the cartridge 104 may support at least one staple pusher 110 and at least one B-form staple 112. The cartridge 104 may be configured to guide the staple pusher 110 and staple 112 toward the anvil 102 and/or staple pocket 108 in response to an actuation, such as a motion of an actuating cam, in a manner substantially as is known in the art.

With continued reference to FIG. 1, the staple 112 may be a B-form staple 112 having a backspan 114, a proximal leg 116, and a distal leg 118. The backspan 114 may have a proximal portion coupled to the proximal leg 116, a distal portion coupled to the distal leg 118, and a central portion therebetween.

The staple pocket 108 may be configured to receive both the proximal leg 116 and the distal leg 118 of the B-form staple 112 when the staple 112 is moved toward the anvil 102.

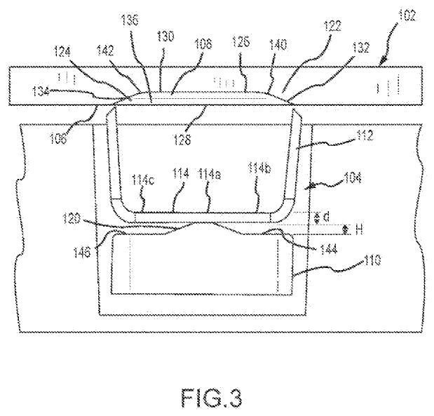

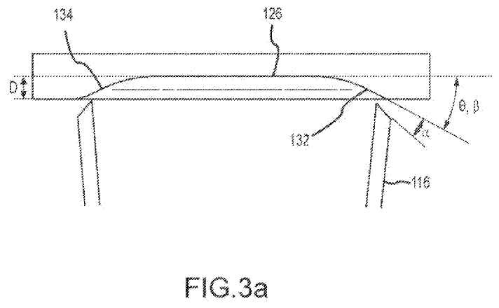

Turning now to FIG. 3, in some embodiments, the staple pusher 110 has a protrusion 120 configured to contact the central portion 114a of the backspan 114 of the B-form staple 112 first. That is, in some embodiments, the staple 112 is supported prior to actuation by the protrusion 120 at the central portion 114a and one or more guides (not illustrated) in the cartridge 104. For the purpose of this document, the central portion 114a of the backspan 114 is understood to be a portion of the backspan 114 that is between a proximal portion 114b and a distal portion 114c, and not necessarily an exact center of the backspan 114.

In some embodiments, the protrusion 120 is configured to contact the central portion 114a of the backspan 114 prior to the staple pusher 110 contacting the proximal portion 114b and the distal portion 114c of the backspan 114.

In some embodiments, in response to the actuation, the staple pusher 110 is configured to displace a central portion 114a of the backspan 114 more than the staple pusher 110 displaces a proximal portion 114b. In some embodiments, in response to the actuation, the staple pusher 110 is configured to displace a central portion 114a of the backspan 114 more than the staple pusher 110 displaces a distal portion 114c.

In some embodiments, the stapler is configured to cold work at least a portion of the backspan 114 during staple deployment. In some embodiments, the staple pusher 110 is configured to cold work at least a portion of the backspan 114. In some embodiments, coldworking is achieved by deforming at least a portion of the backspan 114 during staple deployment, In some embodiments, the staple pusher 110 is configured to displace the central portion 114a of the backspan 114 a more than it does a proximal and/or distal portion 114b, 114c. The displacement of the central portion 114a may be a distance H greater than the proximal and distal portions 114b, 114c. The distance H may be about the same as the thickness or diameter d of the backspan 114 of the staple 112. The distance H may be about the same as the thickness of a wire used to form the staple 112. In some embodiments, the distance H may be between half the thickness or diameter d of the backspan 114 and 1.5 times the thickness or diameter d of the backspan 114. In some embodiments, the distance H may be as much as 25 percent of a length of the staple leg 116, 118. In some embodiments, the distance H may be as much as 50 percent of a gap (not shown) between the anvil 102 and the cartridge 104 during staple deployment (e.g, tissue gap).

In some embodiments, the staple pusher 110 is configured to cold work a portion of a longitudinal length of the backspan 114 of the staple 112. In some embodiments, the staple pusher 110 is configured to cold work a majority of a longitudinal length of the backspan 114 during staple deployment.

In some embodiments, the distance H may be about 0.006 inches. In some embodiments, the distance H may be between 0.004 inches and 0.010 inches, In some embodiments, the distance H may be about 0.008 inches.

In some embodiments, the distance H is configured to reduce a resulting space between the legs 116, 118 and backspan 114 after staple formation, as compared to traditional B-form staples.

Coldworking the backspan 114 and legs 116, 118 during staple deployment may increase the pressure required to force leakage at the tissue closure when compared to the prior art. That is, coldworking the backspan 114 along with the staple legs 116, 118 during staple deployment, the performance of the staple 112 may be further improved. In some embodiments, coldworking the backspan 114 and legs 116, 118 may provide a greater unwinding force when compared to similarly-sized staples currently on the market.

In some embodiments, a surgical stapler is provided having B-form staples made from wire having a diameter of between about 0.005 inches (0.127 millimeters) and about 0.007 inches (0.1778 millimeters). In some embodiments, a surgical stapler or staple is provided, wherein the staple is formed from wire having a diameter of 0.18 millimeters or less. In some embodiments, a surgical stapler or staple is provided, wherein the staple has a backspan thickness of 0.18 millimeters or less. In some embodiments, a surgical stapler is provided having staples made from wire having a diameter of about 0.006 inches (0.1524 millimeters). The staples may be B-form staples. Those skilled in the art will recognize that the wire in these embodiments is significantly smaller than in currently-available staplers. Historically, staples made from wire having a diameter of about 0.008 inches (0.2032 millimeters) have been the standard, because it is believed in the industry that this wire size achieves optimized closure and leak prevention. The Applicant, however, has discovered that smaller diameter wire provides improved closure and leak prevention results, as demonstrated in subsequent portions of this document, Notably, it is counter intuitive that a smaller diameter wire would provide improved tissue closure and leak pressure results, given that the smaller diameter wire has a lower unwinding force.

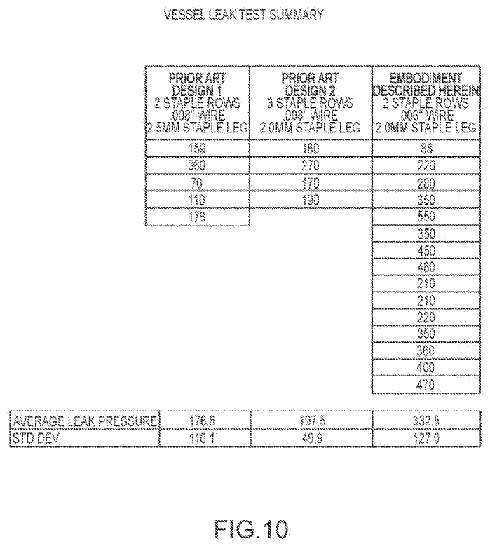

With brief reference to FIG. 10, shown is a table entitled Vessel Leak Test Data. The table illustrates data obtained by testing the performance of Prior Art Design 1, Prior Art Design 2, and Embodiments Described Herein.

The vessel leak test was performed using porcine arteries and veins ranging in thickness between about 0.5 millimeters and about 1 millimeter (splenic, renal and jugular).

The vessels were stapled, divided and mounted to a 27 gauge cannula and clamped proximally on the cannula. A syringe pump infused the vessel with blue dyed water at a rate of 3 cc per minute. A 30 psi pressure gauge was mounted in-line with the test system. Leak pressure was documented at the first signs of blue dye collecting at the staple holes (outside the vessel).

Prior Art Design 1 was tested using two staple rows, as is known in some products currently on the market. The staples in this design were formed from 0.008 inch (0.2032 millimeters) diameter wire and had a leg length of 2.5 millimeters.

Prior Art Design 2 was tested using three staple rows, as is known in some products currently on the market. The staples in this design were formed from 0.008 inch (0.2032 millimeters) diameter wire and had a leg length of 2.0 millimeters.

An embodiment described herein was tested using two staple rows. The staples in this design were formed from 0.006 inch (0.1524 millimeters) diameter wire and had a leg length of 2.0 millimeters.

Performance of embodiments described herein was simulated using a test fixture driven by the Starrett test stand. The anvil included a flat anvil face, and other key anvil features include 0.009 inch (0.2286 millimeters) deep flat pockets and 25 degree lead-ins (proximal and distal surfaces, comparable to surfaces 132, 134) to the pocket. The anvil was positioned 0.015 inches (0.381 millimeters) above the cartridge (i.e., simulating tissue gap) and the pushers were driven 0.005 inches (0.127 millimeters) proud of the cartridge.

As seen in FIG. 10, the average leak pressure of the embodiment described herein, at 332.5 mmHg (millimeters of mercury), was significantly higher than the average leak pressure of the prior art, at 176.6 mmHg and 197.5 mmHg respectively.

Returning reference to FIGS. 2 and 3, in some embodiments, the staple pocket 108 has a proximal portion 122 to receive the proximal leg 116 of the B-form staple 112 and a distal portion 124 to receive the distal leg 118 of the B-form staple 112. A flat surface 126 may extend therebetween.

In some embodiments, the staple pocket 108 has a single recess 128 positioned in the anvil 102. The single recess 128 may have a bottom surface 130 and a plurality of side surfaces 132, 134, 136, 138. The bottom surface 130 may be flat. The bottom surface 130 may be parallel to the face 140 of the anvil 102. A pair of side surfaces 136, 138 may be parallel to each other and/or a direction of travel of the backspan 114 of the B-form staple 112. A pair of longitudinally-extending side surfaces 136, 138 may limit the B-form staple 112 to a desired plane relative to the anvil 102 or prevent the B-form staple 112 from rotating out of a desired pocket area.

The plurality of side surfaces 132, 134, 136, 138 may include a proximal guide surface 132 for guiding the proximal leg 116 of the B-form staple 112. The plurality of side surfaces 132, 134, 136, 138 may include a distal guide surface 134 for guiding the distal leg 118 of the B-form staple 112. The proximal guide surface 132 may be at a non-parallel and non-perpendicular angle Q relative to the face 140. The proximal guide surface 132 may be at a non-parallel and non-perpendicular angle relative to the backspan 114 of the staple 112. The proximal guide surface 132 may be at a non-parallel and non-perpendicular angle b relative to the bottom surface 130 of the staple pocket 108. As illustrated, the bottom surface 130 is parallel to the face 140; therefore, the angles Q, b are the same in the embodiment shown, though those skilled in the art will recognize this is not necessarily a requirement.

The distal guide surface 134 may be at a non-parallel and non-perpendicular angle relative to the face 140. The distal guide surface 134 may be at a non-parallel and non-perpendicular angle relative to the backspan 114 of the staple 112. The distal guide surface 134 may be at a non-parallel and non-perpendicular angle relative to the bottom surface 130 of the staple pocket 108. The angles may be the same or different from those described in reference to the proximal guide surface 132.

In some embodiments, the angle(s) Q, b may be between 20 degrees and 40 degrees. In some embodiments, the angle(s) Q, b may be between 25 degrees and 35 degrees. In some embodiments, the angle(s) Q, b may be 30 degrees.

The proximal guide surface 132 and the distal guide surface 134 may both be flat and may form an obtuse angle therebetween.

In some embodiments, one or both staple legs 116, 118 may include chisel tips 116a, 118a. That is, the legs 116, 118 may be cut at an angle relative to the axis of the respective legs 116, 118, Those skilled in the art will know that the current industry standard is to provide a 45 degree angle between the surface of the tips 116a, 118a and the longitudinal axis of the legs 116, 118.

In some embodiments, and as most clearly seen with brief reference to FIG. 3a, an angle a between the chisel tip 116a, 118a and the guide surface 132, 134 may be provided to promote bending or curving of the staple leg 116, 118 during actuation. The angle a may be less than 50 degrees. The angle a may be less than 40 degrees. The angle a may be between 0 degrees and 5 degrees and the staple 112 may include a feature, such as a notch, to promote bending in a desired direction, such as the features and staples described in co-owned and co-pending application 62/781,074, filed on Dec. 18, 2018, the entire disclosure of which is incorporated herein by reference for all proper purposes. The angle a may be less than 20 degrees. The angle a may be less than 10 degrees. The angle a may be greater than 10 degrees. The angle a may be greater than 15 degrees. The angle a may be greater than 20 degrees. The angle a may be about 30 degrees. The angle a may be between 20 degrees and 45 degrees. The angle a may be between 25 degrees and 35 degrees.

The staple pocket 108 may include a curved surface 141 between the proximal guide surface 132 and the bottom surface 130. The staple pocket 108 may include a curved surface 142 between the distal guide surface 134 and the bottom surface 130.

The bottom surface 130 and at least two 132, 134 of the plurality of the side surfaces may cause the proximal and distal legs 116, 118 of the B-form staple 112 to bend as the staple 112 approximates the anvil 102 or staple pocket 108, such as during staple deployment.

Briefly referencing FIG. 3b, in some embodiments, a depth D of the staple pocket 108 is selected to prevent the staple 112 from tilting and/or falling during staple deployment. In some embodiments, the depth D is at least half the thickness or diameter d of the wire forming the staple 112 or the backspan 114 of the staple 112. In some embodiments, the depth D is at least the thickness or diameter d of the wire forming the staple 112 or the backspan 114 of the staple 112. In some embodiments, the depth D is between 0.5 the thickness or diameter d and 2 times the thickness or diameter d. In some embodiments, the depth D is up to 2 times the thickness or diameter d.

In some embodiments, the depth D of the staple pocket 108 is less than half the thickness or diameter d of the wire forming the staple 112 or the backspan 114 of the staple 112.

With continued reference to FIG. 3, in some embodiments, the staple pusher 110 has a proximal contact portion 144 positioned proximal of the protrusion 120. The proximal contact portion 144 may be configured to contact the backspan 114 of the B-form staple 112 after the protrusion 120 contacts the backspan 114 during staple deployment. In some embodiments, the staple pusher 110 has a distal contact portion 146 positioned distal of the protrusion 120. The distal contact portion 146 may be configured to contact the backspan 114 of the B-form staple 112 after the protrusion 120 contacts the backspan 114 during staple deployment.

In some embodiments, the staple pocket 108 and staple pusher 110 are configured to cold work a majority of the material making up the staple 112.

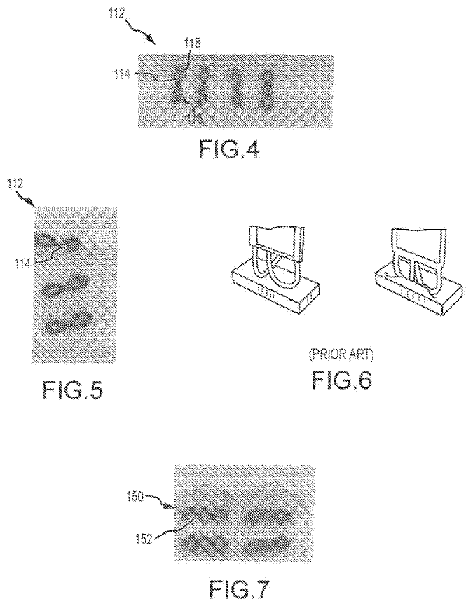

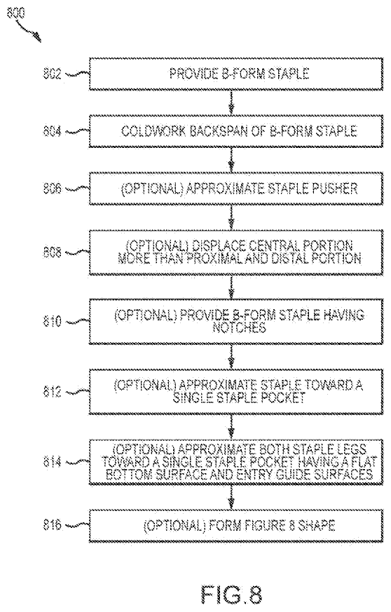

Turning now to FIG. 4, some embodiments described herein are configured to cause a B-form staple 112 to form a Figure 8 shape about tissue. For example, a staple pusher 110 and staple pocket 108 may be configured to form a Figure 8 shape from a standard B-form staple 112. For the purpose of this document, the term “Figure 8” when used to reference a deployed or coldworked staple shall be understood to include an imperfect Figure 8 shape and a B-shape having a collapsed backspan.

Turning now to FIG. 5, shown is a known B-form staple 112 after deployment with a “traditional” staple pocket arrangement and a staple pusher 110 as described herein. Note the relative increase in staple material that is coldworked in embodiments herein (see the backspan 114), as contrasted with the formed staples (prior art) illustrated in FIG. 6.

Turning now to FIG. 7, shown is a set of B-form staples having notches therein, after deployment, demonstrating that, all other factors being the same, the notches in the staples can be used to optimize staple formation. Some embodiments described herein result in a particularly desirable staple formation when used with a B-form staple 150 as described in U.S. Pat. Appin. No. 62/781,074, previously incorporated herein by reference. That is, a B-form staple 150 having one or more notches positioned between the staple legs and the backspan 152 may result in a substantially reduced gap between the formed legs and the backspan 152. This reduced gap may improve vessel closure.

With reference now to FIG. 8, a method 800 is described.

The method 800 may include providing 802 a B-form staple and coldworking 804 the backspan of the B-form staple. Forming 804 may include approximating 806 a staple pusher 110 toward the staple, Forming 804 may include displacing 808 a central portion 114a of a backspan of the staple more than proximal and distal portions of the backspan 114. Providing 802 may include providing 810 a B-form staple having notches between the backspan and the staple legs. Forming 804 may include forming 816 the staple into a figure 8 shape.

The method 800 may include approximating 812 the B-form staple 112 toward a single staple pocket 108. The method 800 may include approximating 814 both staple legs of the staple toward a single staple pocket having a flat bottom surface and a pair of entry guide surfaces.

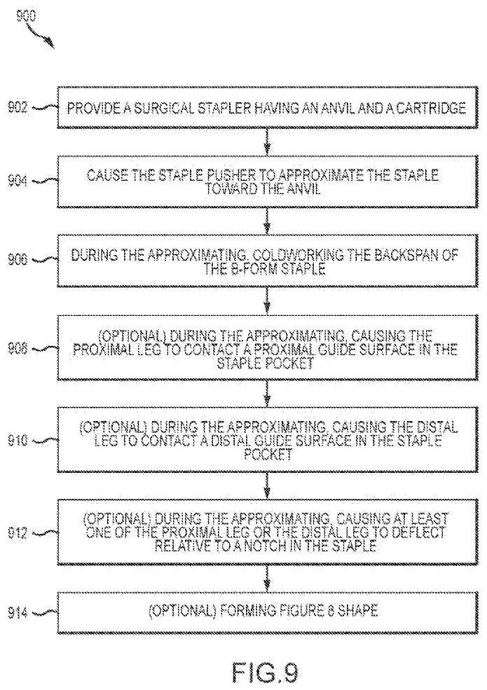

With reference now to FIG. 9, an exemplary method 900 is now described.

The method 900 may include providing 902 a surgical stapler having an anvil and a cartridge, the cartridge supporting a staple pusher and a B-form staple; causing 904 the staple pusher to approximate the staple toward the anvil; and during the approximating, coldworking 906 the backspan of the B-form staple.

The method 900 may include during the approximating, causing 908 the proximal leg to contact a proximal guide surface in the staple pocket.

The method 900 may include, during the approximating, causing 910 the distal leg to contact a distal guide surface in the staple pocket.

The method 900 may include, during the approximating, causing 912 at least one of the proximal leg or the distal leg to deflect relative to a notch in the staple.

The method 900 may include causing 914 the B-form staple to form a Figure-8 shape during deployment.

The following clauses set out further illustrative aspects useful for understanding the appended claims:

Clause (1) A surgical stapler configured to apply a surgical staple to tissue, the surgical stapler comprising: an anvil having a staple pocket; and a cartridge, the cartridge supporting a staple pusher and a B-form staple, the B-form staple having a backspan, a proximal leg, and a distal leg, the backspan having a proximal portion coupled to the proximal leg, a distal portion coupled to the distal leg, and a central portion therebetween; wherein the surgical stapler is configured to cold work at least a portion of the backspan during a deployment of the surgical staple.

Clause (2) The surgical stapler of clause 1, wherein: the surgical stapler is configured to cold work a majority of the backspan during the deployment.

Clause (3) The surgical stapler of clause 1, wherein: the staple pusher is configured to deform the central portion of the backspan of the staple during the deployment.

Clause (4) The surgical stapler of clause 1, wherein: the surgical stapler is configured to form the B-form staple into a Figure-8 shape during the deployment.

Clause (5) The surgical stapler of clause 1, wherein: during the deployment, the staple pusher is configured to contact the central portion of the backspan of the B-form staple prior to contacting the proximal portion and the distal portion of the backspan of the B-form staple.

Clause (6) The surgical stapler of clause 1, wherein: the staple pocket is configured to receive both the proximal leg and the distal leg of the B-form staple when the B-form staple is moved toward the anvil.

Clause (7) The surgical stapler of clause 6, wherein the staple pocket comprises: a single recess formed in the anvil, the single recess having a proximal portion to receive the proximal leg of the B-form staple, a distal portion to receive the distal leg of the B-form staple, and a recessed flat surface therebetween.

Clause (8) The surgical stapler of clause 1, wherein the staple pocket comprises: a single recess positioned in the anvil, the single recess having a bottom surface and a plurality of side surfaces; wherein the bottom surface is substantially flat.

Clause (9) The surgical stapler of clause 8, wherein: the plurality of side surfaces comprises a proximal guide surface for guiding the proximal leg of the B-form staple and a distal guide surface for guiding the distal leg of the B-form staple.

Clause (10) The surgical stapler of clause 8, wherein: the bottom surface and at least two of the plurality of the side surfaces cause the proximal and distal legs of the B-form staple to bend as the B-form staple is moved toward the anvil.

Clause (11) The surgical stapler of clause 8, wherein: the backspan of the staple has a vertical thickness; and the single recess has a depth of between one-half the thickness and two times the thickness.

Clause (12) The surgical stapler of clause 11, wherein: the depth is at least as great as the thickness.

Clause (13) The surgical stapler of clause 8, wherein: the backspan of the staple has a vertical thickness; and the single recess has a depth of less than one-half the thickness.

Clause (14) The surgical stapler of clause 1, wherein the staple pusher comprises: a proximal contact portion positioned proximal of the protrusion; wherein the proximal contact portion is configured to contact the backspan of the B-form staple after the protrusion contacts the backspan of the B-form staple as the cartridge and anvil are moved toward the approximated position.

Clause (15) The surgical stapler of clause 1, wherein the staple pusher comprises: a distal contact portion positioned distal of the protrusion; wherein the distal contact portion is configured to contact the backspan of the B-form staple after the protrusion contacts the backspan of the B-form during the deployment.

Clause (16) The surgical stapler of clause 1, wherein: the staple is made of a wire having a diameter of about 0.006 inches.

Clause (17) A surgical stapler, comprising: a cartridge housing a plurality of B-form staples, the plurality of B-form staples formed from wire having a diameter of between 0.005 inches or 0.127 millimeters and 0.007 inches or 0.1778 millimeters; and an anvil; wherein the anvil and the cartridge are configured to cooperate to form the plurality of B-form staples about tissue.

Clause (18) A surgical stapler, comprising: a cartridge housing a plurality of B-form staples, the plurality of B-form staples formed from wire having a diameter of 0.18 millimeters or less; and an anvil; wherein the anvil and the cartridge are configured to cooperate to form the plurality of B-form staples about tissue.

Clause (19) The surgical stapler of clause 17 or 18, wherein: the plurality of B-form staples are formed from wire having a diameter of about 0.006 inches or 0.1524 millimeters.

Clause (20) The surgical stapler of any one of clause 17-19, wherein: the cartridge houses the plurality of B-form staples in four rows, with a first two of the four rows on a first side of a channel for a tissue cutting mechanism and a second two of the four rows on a second side of the channel.

Clause (21) A method, comprising: providing a surgical stapler having an anvil and a cartridge, the cartridge supporting a staple pusher and a B-form staple, the B-form staple having a backspan and a pair of legs extending therefrom; causing the staple pusher to approximate the staple toward the anvil; and during the approximating, coldworking the backspan of the staple.

Clause (22) The method of clause 20, comprising: causing the B-form staple to form a Figure-8 shape.

Clause (23) The method of clause 20 or 21, comprising: during the approximating, causing the proximal leg to contact a proximal guide surface in the staple pocket; and during the approximating, causing the distal leg to contact a distal guide surface in the staple pocket.

Clause (24) The method of any one of clause 20 through 23, comprising: during the approximating, causing at least one of the proximal leg or the distal leg to deflect relative to a notch in the staple.

Clause (25) A surgical staple suitable for use with the stapler or method of any clause herein, wherein the staple has a backspan thickness of 0.18 millimeters or less.

Clause (26) An anvil for a surgical stapler, as described anywhere herein.

Clause (27) A staple pusher for a surgical stapler, as described anywhere herein.

Clause (28) A staple for a surgical stapler, as described anywhere herein.

Each of the various elements disclosed herein may be achieved in a variety of manners, This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, the disclosure of a “fastener” should be understood to encompass disclosure of the act of “fastening”—whether explicitly discussed or not—and, conversely, were there only disclosure of the act of “fastening”, such a disclosure should be understood to encompass disclosure of a “fastening mechanism”. Such changes and alternative terms are to be understood to be explicitly included in the description.

Moreover, the claims shall be construed such that a claim that recites “at least one of A, B, or C” shall read on a device that requires “A” only. The claim shall also read on a device that requires “B” only. The claim shall also read on a device that requires “C” only.

Similarly, the claim shall also read on a device that requires “A+B”. The claim shall also read on a device that requires “A+B+C”, and so forth.

The claims shall also be construed such that any relational language (e.g. perpendicular, straight, parallel, flat, etc.) is understood to include the recitation “within a reasonable manufacturing tolerance at the time the device is manufactured or at the time of the invention, whichever manufacturing tolerance is greater”.

Those skilled in the art can readily recognize that numerous variations and substitutions may be made in the invention, its use and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

1. An end effector configured for engagement with a surgical stapler actuator, the end effector comprising:
an anvil having a staple pocket; and
a cartridge, the cartridge supporting a staple pusher and configured to house a staple, the staple having a backspan, a proximal leg, and a distal leg, the backspan having a proximal portion coupled to the proximal leg, a distal portion coupled to the distal leg, and a central portion therebetween;
wherein the staple pusher has a protrusion configured to cold work the central portion of the backspan during a deployment of the staple by displacing the central portion of the backspan of the staple relative to the proximal and distal portions of the backspan by a height that is between 0.5 and 1.5 times a thickness of the backspan of the staple.

2. The end effector of claim 1, wherein the staple pusher is configured to cold work a majority of the backspan during the deployment.

3. The end effector of claim 1, wherein the stapler pocket is configured to form the staple into a Figure-8 shape.

4. The end effector of claim 1, wherein during the deployment, the staple pusher is configured to contact the central portion of the backspan of the staple prior to contacting the proximal portion and the distal portion of the backspan of the staple.

5. The end effector of claim 1, wherein the staple pocket is configured to receive both the proximal leg and the distal leg of the staple when the staple is moved toward the anvil.

6. The end effector of claim 5, wherein the staple pocket comprises a recess formed in the anvil, the recess having a proximal portion to receive the proximal leg of the staple, a distal portion to receive the distal leg of the staple, and a recessed surface therebetween.

7. The end effector of claim 1, wherein the staple pocket comprises a recess positioned in the anvil, the recess having a bottom surface and a plurality of side surfaces, and wherein the bottom surface is substantially flat.

8. The end effector of claim 7, wherein the plurality of side surfaces comprises a proximal guide surface for guiding the proximal leg of the staple and a distal guide surface for guiding the distal leg of the staple.

9. The end effector of claim 7, wherein the bottom surface and at least two of the plurality of the side surfaces are configured to cause the proximal and distal legs of the staple to bend as the staple is moved toward the anvil.

10. The end effector of claim 7, wherein the backspan of the staple has a vertical thickness, and wherein the recess of the staple pocket has a depth of between one-half the vertical thickness and two times the vertical thickness of the backspan of the staple.

11. The end effector of claim 10, wherein the depth of the recess of the staple pocket is at least as great as the vertical thickness of the backspan of the staple.

12. The end effector of claim 7, wherein the backspan of the staple has a vertical thickness, and wherein the recess of the staple pocket has a depth of less than one-half the thickness of the backspan of the staple.

13. The end effector of claim 1, wherein the staple pusher comprises a proximal contact portion positioned proximal to the protrusion; and
wherein the proximal contact portion of the staple pusher is configured to contact the backspan of the staple after the protrusion of the staple pusher contacts the backspan of the staple during the deployment.

14. The end effector of claim 1, wherein the staple pusher comprises a distal contact portion positioned distal to the protrusion; and
wherein the distal contact portion of the staple pusher is configured to contact the backspan of the staple after the protrusion of the staple pusher contacts the backspan of the staple during the deployment.

15. The end effector of claim 1, wherein the cartridge is configured to house a staple formed from a wire having a diameter of 0.18 millimeters or less.

16. The end effector of claim 1, wherein the cartridge is configured to house a staple formed from a wire having a diameter of about 0.006 inches or 0.1524 millimeters.

17. The end effector of claim 1, wherein the staple pusher comprises a proximal contact portion proximal to the protrusion, and a distal contact portion distal to the protrusion, and wherein the protrusion has a height relative to the proximal portion and the distal portion that is between 0.5 and 1.5 times a thickness of the backspan of the staple.

18. An end effector configured for engagement with a surgical stapler actuator, the end effector comprising:
an anvil having a staple pocket; and
a cartridge, the cartridge supporting a staple pusher and configured to house a staple, the staple having a backspan, a proximal leg, and a distal leg, the backspan having a proximal portion coupled to the proximal leg, a distal portion coupled to the distal leg, and a central portion therebetween;
wherein the staple pusher has (1) a protrusion, (2) a proximal contact portion proximal to the protrusion, and (3) a distal contact portion distal to the protrusion, the proximal contact portion extending from a first side edge of the staple pusher, the distal contact portion extending from a second side edge of the staple pusher, and wherein the proximal contact portion and the distal contact portion of the staple pusher are configured to contact the backspan of the staple after the protrusion of the staple pusher contacts the central portion of the backspan of the staple during a deployment of the staple.

19. The end effector of claim 18, wherein the staple pusher has a protrusion configured to cold work the central portion of the backspan during the deployment of the staple by displacing the central portion of the backspan of the staple relative to the proximal and distal portions of the backspan by a height that is between 0.5 and 1.5 times a thickness of the backspan of the staple.
